(19) United States
(12) Patent Application Publication
(10) Pub. No.: US 2010/0274334 A1
(43) Pub. Date: Oct. 28, 2010

(54) MULTI-ZONE LEAD COATINGS

(76) Inventor: Shrojalkumar Desai, Little Canada, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/708,942

(22) Filed: Feb. 19, 2010

Related U.S. Application Data
(60) Provisional application No. 61/171,505, filed on Apr. 22, 2009.

(51) Int. Cl. A61N 1/05; B05D 7/00

(52) U.S. Cl. 607/116; 427/2.1

(57) ABSTRACT

Multi-zone surface treatments for medical electrical leads are provided for treating an intravenous zone, an intracardiac zone and/or a tip zone of a lead with multiple surface modifications to achieve distinct performance characteristics.

### CROSS-REFERENCE TO RELATED APPLICATION

This application claims the benefit under 35 U.S.C. §119 of U.S. Provisional Application No. 61/171,505, filed on Apr. 22, 2009, entitled “Multi-Zone Lead Coatings,” which is incorporated herein by reference in its entirety for all purposes.

### TECHNICAL FIELD

The present invention relates to medical electrical lead devices and methods for accessing an anatomical space of the body. More specifically, the invention relates to surface treatments for lead components.

### BACKGROUND

Various types of medical electrical leads for use in cardiac rhythm management (CRM) and neurostimulation systems are known. For CRM systems, such leads are typically extended intravascularly to an implantation location within or on a patient's heart, and thereafter coupled to a pulse generator or other implantable device for sensing cardiac electrical activity, delivering therapeutic stimuli, and the like. The leads are desirably highly flexible to accommodate natural patient movement, yet also constructed to have minimized profiles. At the same time, the leads are exposed to various external forces imposed, for example, by the human muscular and skeletal system, the pulse generator, other leads, and surgical instruments used during implantation and explantation procedures.

### SUMMARY

In Example 1, a medical electrical lead comprises: a flexible, elongated polymeric lead body defining at least one longitudinal lumen therethrough; at least one conducting wire extending through the at least one lumen; a connector coupled to the lead body for mechanically and electrically coupling the lead to an implantable pulse generator device; and an electrode disposed on an exterior portion of the lead body, wherein electrode is electrically coupled to the conducting wire. The lead has an outer surface including at least a distal tip zone, a medial intracardiac zone and a proximal intravenous zone. The outer surface includes at least two surface modifications. At least two of the zones include a surface modification; and at least two surface modifications are selected from a first surface modification comprising a polymeric coating material including a glyme, a second surface modification comprising a silazane, a third surface modification comprising a fluorocarbon and a fourth surface modification comprising a NiPAM polymer coating.

In Example 2, the medical electrical lead of Example 1 wherein the outer surface of the lead includes at least three surface modifications.

In Example 3, the medical electrical lead of Example 1, wherein the intravenous zone includes at least the first surface modification, the intracardiac zone includes at least one of the first, second or third surface modifications and the tip zone includes at least the third surface modification.

In Example 4, the medical electrical lead of Example 1, further comprising a pocket zone and wherein the pocket zone includes at least one of the first, second, third or fourth surface treatments.

In Example 5, the medical electrical lead of Example 1, wherein the polymeric coating material of the first surface modification comprises a tetraglyme.

In Example 6, the medical electrical lead of Example 1, wherein the silazane comprises a hexamethyldisilazane treatment.

In Example 7, the medical electrical lead of Example 1, wherein the fluorocarbon comprises a tetrafluoromethane treatment, a hexafluoroethane treatment, an octofluoropropane treatment, an octofluorocyclobutane treatment or a hexafluorocyclopropane treatment.

In Example 8, the medical electrical lead of Example 1, wherein the NiPAM coating comprises cell adhesive proteins, peptides, peptide fragments or a combination thereof.

In Example 9, a process for modifying an outer surface of a medical electrical lead having a proximal intravenous zone, a medial intracardiac zone and a distal tip zone comprises: treating a portion of at least one zone with at least one surface modification selected from a polymeric coating material including a glyme, a silazane treatment, a fluorocarbon treatment and a NiPAM polymer coating material; and treating a portion of at least a second zone with at least one of the surface modifications, wherein the outer surface of the lead is treated with at least two different surface modifications.

In Example 10, the process of Example 9, further comprising the step of coating at least a portion of the intravenous zone with the polymeric material including a glyme.

In Example 11, the process of Example 9, further comprising the step of contacting at least a portion of the intracardiac zone with a silazane, fluorocarbon or both.

In Example 12, the process of Example 9, further comprising the step of coating at least a portion of the tip zone with the NiPAM polymer coating.

In Example 13, the process of Example 9, wherein at least one of the treating steps comprises a plasma deposition treatment, a spray coating treatment or a dip coating treatment.

In Example 14, the process of Example 9, wherein at least one coating material is applied to a portion of the outer surface and wherein micro- or nano-patterns are formed in the coated portion.

In Example 15, the process of Example 9, wherein each treatment step comprises a plasma deposition treatment.

In Example 16, the process of Example 9, further comprising the step of treating each of the proximal intravenous zone, medial intracardiac zone and distal tip zone with at least one of the surface modifications.

In Example 17, a process for modifying an outer surface of a medical electrical lead having a proximal intravenous zone, a medial intracardiac zone and a distal tip zone comprises: coating at least a portion of the intravenous zone with a first polymeric coating including a glyme; contacting at least a portion of the intracardiac zone with the first polymeric material, silazane, fluorocarbon or a combination; and coating at least a portion of the tip zone with a second polymeric coating including a NiPAM polymer.

In Example 18, the process of Example 17, wherein the first polymeric coating comprises a tetraglyme.

In Example 19, the process of Example 17, wherein the contacting step comprises contacting the intracardiac zone with hexamethyldisilazane.

In Example 20, the process of Example 17, wherein the contacting step comprises contacting the intracardiac zone with CF₄, C₂F₆ or C₃F₈.

In Example 21, the process of Example 17, wherein the second polymeric coating comprises a copolymer of NiPAM and hydroxyethyl methacrylate, ethylene glycol, polyvinyl pyrrolidone, cell adhesive biomolecules or a combination of the foregoing.

In Example 22, the process of Example 17, further comprising the step of modifying at least a portion of a pocket zone proximal to the intravenous zone with at least one of the coating or contacting steps.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic drawing of a cardiac rhythm management system including a pulse generator coupled to a pair of medical electrical leads deployed in a patient's heart, according to one embodiment.

FIG. 2 is a perspective view of one of the leads shown in FIG. 1, according to one embodiment.

FIG. 3 shows bar graphs relating to tissue adsorption and adhesion characteristics of various materials.

FIG. 4 shows comparative images of human cellular growth in tissue cultures.

FIG. 5 is an IR spectroscopy result for fluorocarbon treatments.

FIG. 6 are comparative images relating to tissue layer adhesion.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

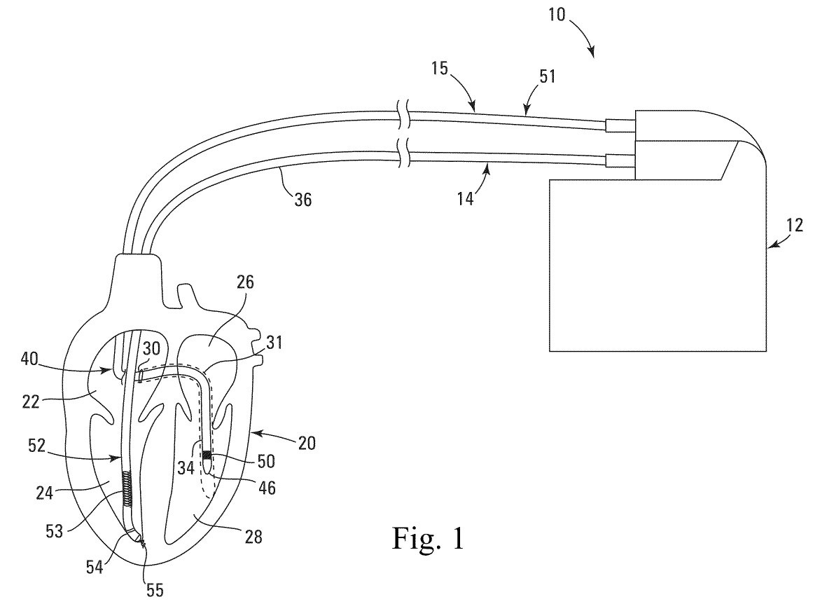

The various embodiments disclosed herein relate to a medical electrical lead having multi-zone surface treatments and related methods of making the lead. The leads according to the various embodiments of the present invention are suitable for sensing intrinsic electrical activity and/or applying therapeutic electrical stimuli to a patient. Exemplary applications include, without limitation, cardiac rhythm management (CRM) systems and neurostimulation systems. For example, in exemplary CRM systems utilizing pacemakers, implantable cardiac defibrillators, and/or cardiac resynchronization therapy (CRT) devices, the medical electrical leads according to embodiments of the invention can be endocardial leads configured to be partially implanted within one or more chambers of the heart so as to sense electrical activity of the heart and apply a therapeutic electrical stimulus to the cardiac tissue within the heart. Additionally, the leads formed according to embodiments of the present invention may be particularly suitable for placement in a coronary vein adjacent to the left side of the heart so as to facilitate bi-ventricular pacing in a CRT or CRT-D system. Still additionally, leads formed according to embodiments of the present invention may be configured to be secured to an exterior surface of the heart (i.e., as epicardial leads). FIG. 1 is a schematic drawing of a cardiac rhythm management system 10 including a pulse generator 12 coupled to a pair of medical electrical leads 14, 15 deployed in a patient's heart 20, which includes a right atrium 22 and a right ventricle 24, a left atrium 26 and a left ventricle 28, a coronary sinus ostium 30 in the right atrium 22, a coronary sinus 31, and various coronary veins including an exemplary branch vessel 34 off of the coronary sinus 31.

According to one embodiment, as shown in FIG. 1, lead 14 includes a proximal portion and a distal portion 40, which as shown is guided through the right atrium 22, the coronary sinus ostium 30 and the coronary sinus 31, and into the branch vessel 34 of the coronary sinus 31. The distal portion 40 further includes a distal end 46 and an electrode 50 both positioned within the branch vessel 34. The illustrated position of the lead 14 may be used for delivering a pacing and/or defibrillation stimulus to the left side of the heart 20. Additionally, it will be appreciated that the lead 14 may also be partially deployed in other regions of the coronary venous system, such as in the great cardiac vein or other branch vessels for providing therapy to the left side or right side of the heart 20.

In the illustrated embodiment, the electrode 50 is a relatively small, low voltage electrode configured for sensing intrinsic cardiac electrical rhythms and/or delivering relatively low voltage pacing stimuli to the left ventricle 28 from within the branch coronary vein 34. In various embodiments, the lead 14 can include additional pace/sense electrodes for multi-polar pacing and/or for providing selective pacing site locations.

As further shown in the illustrated embodiment, the lead 15 includes a proximal portion 51 and a distal portion 52 implanted in the right ventricle 24. In other embodiments, the CRM system 10 may include still additional leads, e.g., a lead implanted in the right atrium 22. The distal portion 52 further includes a flexible, high voltage electrode 53 and a relatively low-voltage ring electrode 54. As will be appreciated, the high voltage electrode 53 has a relatively large surface area compared to the ring electrode 54, and is thus configured for delivering relatively high voltage electrical stimulus to the cardiac tissue for defibrillation/cardioversion therapy, while the ring electrodes 54 is configured as a relatively low voltage pace/sense electrode. Fixation device 55 may be used to fix the distal lead tip to the heart or vessel wall.

In various embodiments, the lead 15 includes additional defibrillation/cardioversion and/or additional pace/sense electrodes positioned along the lead 15 so as to provide multi-polar defibrillation/cardioversion capabilities. In one exemplary embodiment, the lead 15 includes a proximal high voltage electrode in addition to the electrode 53 positioned along the lead 15 such that it is located in the right atrium 22 (and/or superior vena cava) when implanted. As will be appreciated, additional electrode configurations can be utilized with the lead 15. In short, any electrode configuration can be employed in the lead 16 without departing from the intended scope of the present invention.

The pulse generator 12 is typically implanted subcutaneously within an implantation location in the patient's chest or abdomen. The pulse generator 12 may be any implantable medical device known in the art or later developed, for delivering an electrical therapeutic stimulus to the patient. In various embodiments, the pulse generator 12 is a pacemaker, an implantable cardiac defibrillator, a cardiac resynchronization (CRT) device configured for bi-ventricular pacing, and/or includes combinations of pacing, CRT, and defibrillation capabilities.

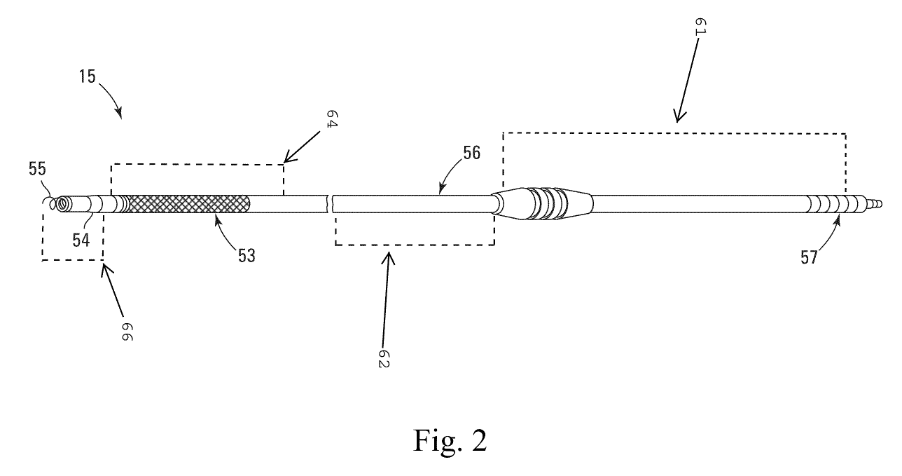

FIG. 2 is a perspective view of the lead 15 shown in FIG. 1. Of course, the same concepts could be applied to lead 14 or any other type of CRM or neurostimulation lead. As discussed above, the lead 15 is adapted to deliver electrical pulses to stimulate a heart and/or for receiving electrical pulses to monitor the heart. The lead 15 includes an elongated polymeric lead body 56, which may be formed from any polymeric material such as polyurethane, silicone rubber, or the like.

As further shown, the lead 15 further includes a connector 57 operatively associated with the proximal end of the lead body 56. The connector 57 is configured to mechanically and electrically couple the lead 15 to the pulse generator 12, and may be of any standard type, size or configuration. The connector 57 is electrically and mechanically connected to the electrodes 53, 54, 55 by way of one or more conducting wires (not shown) within the lead body 56. The conducting wires utilized may take on any configuration providing the necessary functionality. For example, the conducting wires coupling the electrodes 54 and/or 55 to the connector 57 (and thus, to the pulse generator 12) may be coiled conductors defining an internal lumen for receiving a stylet or guidewire for lead delivery. Conversely, in various embodiments, the conducting wire to the high voltage electrode 53 may be a multi-strand cable conductor.

The outer surface of the lead 15 shown in FIG. 2 includes multiple zones, the approximate boundaries of which are illustrated by dashed lines. These zones include a pocket zone 61, an intravenous zone 62, an intracardiac zone 64, and a tip zone 66. The pocket zone 61 represents portions of the lead that reside in vessels somewhat distant from the heart. The intravenous zone 62 generally represents portions of the lead that reside in vessels that lead to the heart. The intracardiac zone 64 generally represents portions of the lead that reside within the heart, and generally includes one or more electrodes. The tip zone 66 generally represents the distal portion of the lead that may include passive or active lead fixation devices such as tines and helical screws. The zones illustrated in FIG. 2 may vary in length and/or position on the lead depending on the type and size of the lead, the intended treatment and the intended implantation procedure.

In one embodiment, one or more of these zones includes at least one surface modification, and in one embodiment, at least two different surface modifications. In another embodiment, at least two surface modifications are used to treat at least three zones. These surface modifications may be optimized with respect to each zone to provide desired lead performance characteristics.

A first surface modification is a polymeric coating material exhibiting a number of desirable characteristics, including non-fouling, non-thrombogenic and wet lubricity characteristics. As such, the first coating material may be particularly suitable for use in the intravenous zone 62 and/or intracardiac zone 64. The first coating material may also be suitable for treating additional lead zones and or lead components, including the pocket zone 61 and the pulse generator (not shown).

In one embodiment, the polymeric coating material of the first surface modification is a glycol ether commonly referred to as a glyme. Suitable glymes include monoglyme, ethyl glyme, diglyme, ethyl diglyme, triglyme, butyl diglyme, tetraglyme, pentaglyme, hexaglyme and their corresponding mono alkyl ethers. According to another embodiment, these glymes may be functionalized with, for example, silane.

A tetraglyme having the following formula may be particularly suitable:

—[CH₃O(CH₂CH₂O)4CH₃]—

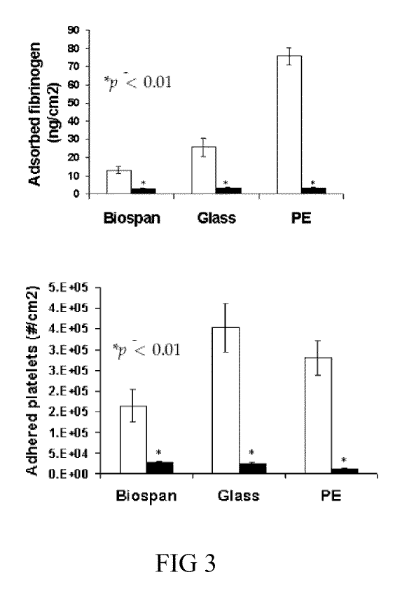

Tetraglyme coated surfaces may exhibit reduced macromolecular absorption and adhesion. FIG. 3 includes two bar graphs showing the results of in vitro cell adhesion tests of various materials. Materials coated with tetraglyme (black bars) exhibit significantly less fibrinogen absorption than non-coated materials (clear bars). Although these tests were not performed on a lead material, a lead body surface coated with such a tetraglyme material may provide similar results.

The second and third surface modifications exhibit different characteristics than the first modification, including in particular low tissue adhesion, thermal stability, environmental stress cracking and metal ion oxidation. As such, these modifications may be particularly suitable for treating the intracardiac zone including the surface of the electrode. Suitable treatment materials for the second and third surface modifications include, for example, silazanes and fluorocarbons.

Silazanes are hydrides of silicon and nitrogen having a straight or branched chain of silicon and nitrogen. A particularly suitable silazane is hexamethyldisilazane, which has the following general formula:

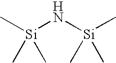

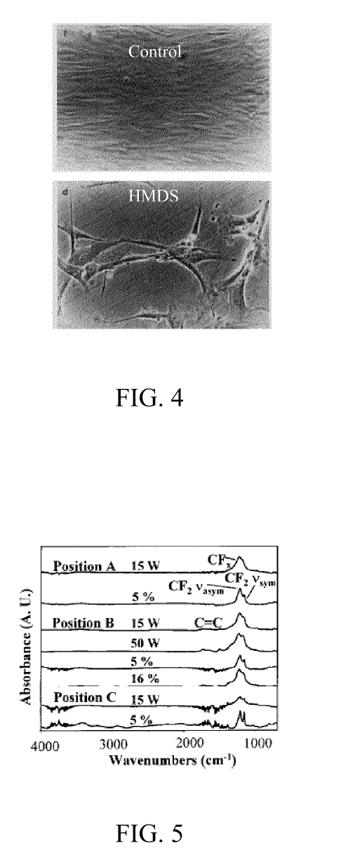

Hexamethyldisilazane (HMDS) is a chemical reagent that may be used in liquid or gas form to treat the surface of the lead. It is commercially available from a number of sources. FIG. 4 is a comparative image of cellular material adhered to a control surface and to an HMDS treated surface. The control exhibits a significant quantity of aligned cellular strands demonstrating confluent cell adhesion. The HMDS treated surface, in contrast, exhibits fewer adhered cells and less strand alignment. Although not performed on a lead body, the results in FIG. 4 indicate that portions or zones of a lead body treated with HMDS in a similar manner may result in reduced adhesion.

Exemplary fluorocarbons that may be used to treat the surface of the lead include fluoroalkyls such as fluoromethanes, fluoroethanes and fluoropropanes. Particularly suitable fluorocarbons include CF₄, C₂F₆ and C₃F₈, which are gasses that can be used to treat substrate surfaces by plasma deposition. Other suitable fluorocarbons include fluorocyclocarbons such as octofluorocyclobutane and hexafluorocyclopropane. FIG. 5 is an IR spectroscopy result indicating that surfaces treated with such fluorocarbon gasses exhibit a generally inert surface similar to PTFE. Although not performed on a lead body, the results indicate that a similar treatment performed on various zones on a lead body may provide a surface mimicking a PTFE.

In one embodiment, both the silazane and fluorocarbon treatments are employed on different zones or zone portions of the lead body. In another embodiment, both are employed at the same zone or zone portion in succession. In a further embodiment, only one of the two modifications is employed.

A fourth modification may be utilized to provide improved lead fixation and tissue in-growth properties, and may be used at the tip zone 66, and particularly on lead fixation structures, including the fixation coil 55, at the distal end of the lead body.

In one embodiment, the fourth modification is a polymeric coating material including a N-isopropyl acrylamide (NiPAM) segment. Certain NiPAM polymers may be particularly suitable because such materials exhibit fixation properties as a function of temperature. For example, certain NiPAM materials promote tissue adhesion at physiological temperature (approximately 37° C.), but are significantly less adhesive at cooler temperatures such as around 25° C. This facilitates lead insertion and removal at lower temperatures and lead fixation at physiological temperature.

Suitable NiPAM polymers may include polymers having NiPAM polymer segments and at least one additional polymer segment such as a poly hydroxyethyl methacrylate (poly HEMA) segment, a polyethylene glycol (PEG) segment and/or a polyvinyl pyrrolidone (PVP) segment. Additionally, suitable NiPAM polymers may incorporate one or more cell and tissue adhesive biomolecules including natural and synthetic proteins/peptides, peptide fragments, amino acid sequences such as Asp-Gly-Glu-Ala (DGEA), Gly-Arg-Gly-Asp (GRGD), GFOGER, or focal adhesion proteins such as talin, α-actinin, filamin, paxillin or vinculin.

In one embodiment the NiPAM polymer is a PEG-PVP-NiPAM tri-block polymer having the following formula:

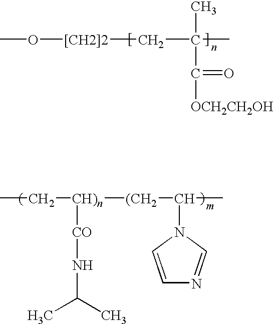

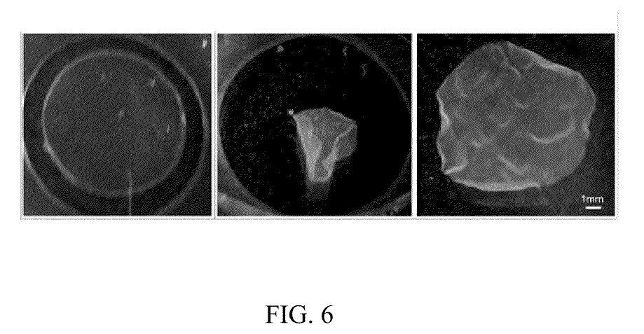

FIG. 6 shows a series of images of a cell layer adhered to a NiPAM material. This image on the left shows a cell monolayer, which at a temperature of 37° C., exhibits high adhesion. The middle picture shows a cell sheet, which at 25° C. exhibits reduced adhesion. The third image shows a cell sheet which remains intact after reducing it to 25° C. and removing from the NiPAM surface. Although not performed on a lead body, the results set forth in FIG. 6 indicate that one or more zones of a lead body coated with a NiPAM polymer may provide similar results.

The surface modifications discussed herein can be carried out according to a variety of methods. The coating materials, for example, can be applied by dip or spray coating as well as by chemical vapor deposition and plasma chemical vapor deposition.

Plasma deposition techniques may be particularly suitable and, in one embodiment, each surface modification is carried out by plasma deposition as part of a lead manufacturing method. In plasma deposition, a plasma is formed by combining the treatment material or precursors thereof with a generally inert carrier gas such as helium or nitrogen, for example. The plasma is delivered to a reaction chamber, and is deposited onto a substrate by charging the plasma with an AC or DC current or an RF signal. For deposition involving coating materials, the coating thickness may be a function of both the strength of the current and the deposition time. Plasma deposition techniques are known to persons of ordinary skill in the art.

According to one method of treating multiple zones by plasma discharge, one or more leads are delivered through multiple plasma reaction chambers, each chamber being adapted to apply a different surface modification. In an alternate embodiment, different lead components or zones are independently treated with a surface modification, and are subsequently combined to form a lead having zones with multiple surface modifications, which have been optimized according to desired performance characteristics of each zone. In a further embodiment, certain portions of the zone are masked prior to coating, or laser treated after coating to provide a micro- or nano-sized pattern in the zone having a desired geometry. The coating materials may also be cured after application by conventional thermal, IR or UV curing techniques.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A medical electrical lead comprising:
a flexible, elongated polymeric lead body defining at least one longitudinal lumen therethrough;
at least one conducting wire extending through the at least one lumen;
a connector coupled to the lead body for mechanically and electrically coupling the lead to an implantable pulse generator device; and
an electrode disposed on an exterior portion of the lead body, wherein the electrode is electrically coupled to the conducting wire;
wherein the lead has an outer surface including at least a distal tip zone, a medial intracardiac zone and a proximal intravenous zone;
wherein the outer surface includes at least two surface modifications;
wherein at least two of the zones include a surface modification; and
wherein at least two surface modifications are selected from
a first surface modification comprising a polymeric coating material including a glyme, a second surface modification comprising a silazane, a third surface modification comprising a fluorocarbon and a fourth surface modification comprising a NiPAM polymer coating.

2. The medical electrical lead of claim 1 wherein the outer surface of the lead includes at least three surface modifications.

3. The medical electrical lead of claim 1 wherein the intravenous zone includes at least the first surface modification, the intracardiac zone includes at least one of the first, second or third surface modifications and the tip zone includes at least the third surface modification.

4. The medical electrical lead of claim 1 further including a pocket zone and wherein the pocket zone includes at least one of the first, second, third or fourth surface treatments.

5. The medical electrical lead of claim 1 wherein the polymeric coating material of the first surface modification comprises a tetraglyme.

6. The medical electrical lead of claim 1 wherein the silazane comprises a hexamethyldisilazane treatment.

7. The medical electrical lead of claim 1 wherein the fluorocarbon comprises a tetrafluoromethane treatment, a hexafluoroethane treatment, an octofluoropropane treatment, an octofluorocyclobutane treatment or a hexafluorocyclopropane treatment.

8. The medical electrical lead of claim 1 wherein the NiPAM coating comprises cell adhesive proteins, peptides, peptide fragments or a combination thereof.

9. A process for modifying an outer surface of a medical electrical lead having a proximal intravenous zone, a medial intracardiac zone and a distal tip zone, the process comprising:
treating a portion of at least one zone with at least one surface modification selected from a polymeric coating material including a glyme, a silazane treatment, a fluorocarbon treatment and a NiPAM polymer coating material;
treating a portion of at least a second zone with at least one of the surface modifications; and
wherein the outer surface of the lead is treated with at least two different surface modifications.

10. The process of claim 9 comprising the step of coating at least a portion of the intravenous zone with the polymeric material including a glyme.

11. The process of claim 9 comprising the step of contacting at least a portion of the intracardiac zone with a silazane, fluorocarbon or both.

12. The process of claim 9 comprising the step of coating at least a portion of the tip zone with the NiPAM polymer coating.

13. The process of claim 9 wherein at least one of the treating steps comprises a plasma deposition treatment, a spray coating treatment or a dip coating treatment.

14. The process of claim 9 wherein at least one coating material is applied to a portion of the outer surface and wherein micro- or nano-patterns are formed in the coated portion.

15. The process of claim 9 wherein each treatment step comprises a plasma deposition treatment.

16. The process of claim 9 further comprising the step of treating each of the proximal intravenous zone, medial intracardiac zone and distal tip zone with at least one of the surface modifications.

17. A process for modifying an outer surface of a medical electrical lead having a proximal intravenous zone, a medial intracardiac zone and a distal tip zone, the process comprising:
coating at least a portion of the intravenous zone with a first polymeric coating including a glyme;
contacting at least a portion of the intracardiac zone with the first polymeric material, silazane, fluorocarbon or a combination; and
coating at least a portion of the tip zone with a second polymeric coating including a NiPAM polymer.

18. The process of claim 17 wherein the first polymeric coating comprises a tetraglyme.

19. The process of claim 17 wherein the contacting step comprises contacting the intracardiac zone with hexamethyldisilazane.

20. The process of claim 17 wherein the contacting step comprises contacting the intracardiac zone with CF₄, C₂F₆ or C₃F₈.

21. The process of claim 17 wherein the second polymeric coating comprises a copolymer of NiPAM and hydroxyethyl methacrylate, ethylene glycol, polyvinyl pyrrolidone, cell adhesive biomolecules or a combination of the foregoing.

22. The process of claim 17 further comprising the step of modifying at least a portion of a pocket zone proximal to the intravenous zone with at least one of the coating or contacting steps.
